(19) United States
(12) Patent Application Publication
(10) Pub. No.: US 2003/0234173 A1
(43) Pub. Date: Dec. 25, 2003

(54) Method and apparatus for treating fluid mixtures with ultrasonic energy

(76) Inventor: Bruce E. Minter, Eagle, ID (US)

Correspondence Address: Joseph W. Holland, P. O. Box 1840, Boise, ID, 83701-1840, US

(21) Appl. No.: 10/176,728

(22) Filed: Jun. 20, 2002

(51) Int. Cl. C07C 1/00

(52) U.S. Cl. 204/157.42; 204/157.62; 422/186

(57) ABSTRACT

A method and apparatus for treating fluid mixtures with ultrasonic energy. In one embodiment, the fluid mixture may include a selected constituent and the method may include directing a flow of the fluid mixture into a treatment apparatus and altering a phase and/or a chemical composition of the selected constituent by exposing the fluid mixture to ultrasonic energy while the fluid mixture flows through the apparatus. In one embodiment, the fluid mixture may be under pressure while being exposed to the ultrasonic energy and the fluid mixture may subsequently be exposed to a vacuum source to remove gas from the fluid mixture. In another aspect of the invention, the ultrasonic energy may have a first frequency and the fluid mixture may be exposed to ultrasonic energy of a second frequency different than the first frequency while in the apparatus. The ultrasonic energy may cavitate a liquid portion of the fluid mixture to generate heat and pressure in the fluid mixture.

### RELATED APPLICATIONS

This application is related to the following application assigned to a common assignee (a) “Ozone Generator”, application Ser. No. 10/123,759 filed Apr. 15, 2002; and the following applications filed concurrently herewith (b) Method and Apparatus for Treating Fluid Mixtures with Ultrasonic Energy; (c) Method and Apparatus for Directing Ultrasonic Energy; (d) and Method and Apparatus for Directing Ultrasonic Energy, which are all herein incorporated by reference.

### BACKGROUND

1. Field of Invention

The present invention relates to methods and apparatuses for treating fluid mixtures, such as agricultural or industrial waste streams, with ultrasonic energy to clean or otherwise alter the waste streams.

2. Background of the Invention

Many industrial, municipal and agricultural processes generate waste matter that is potentially harmful to the environment. Accordingly, a variety of processes have been developed to remove harmful elements from the waste matter before returning the water to lakes, streams and oceans.

Conventional processes include filters, such as reverse osmosis filters that remove solid contaminants from the waste matter. However, because of environmental concerns, it may be difficult to dispose of the solid contaminants removed by the filters. Furthermore, the filters themselves must be periodically back-flushed, which may be a time consuming process.

In an alternate process, microorganisms are disposed in the waste matter to consume or alter harmful elements in the waste matter. However, such systems generally process the waste matter in a batch mode and accordingly may be slow and labor intensive to operate.

Another conventional approach is to sterilize waste matter streams with ultraviolet light. One problem with this approach is that the waste matter must be positioned very close to the light source, which may make ultraviolet systems slow, expensive and inefficient.

Still another method includes exposing the waste matter stream to ozone, which may alter harmful elements in the waste matter stream. One problem with this approach is that the cost of generating effective quantities of ozone is generally so high that the process may not be economically feasible.

Another approach has been to dispose the waste matter in a vessel and apply ultrasonic energy to the waste matter in a batch process. Exposing a fluid mixture, such as a waste matter stream, to ultrasonic energy may cause chemical and/or physical changes to occur in the mixture. For instance, cavitation of a liquid portion of the mixture and generation of heat may occur. Cavitation bubbles formed in the waste matter stream may grow in a cyclic fashion and ultimately collapse. This process creates very high temperatures, pressures, and thermal cycling rates. For example, it is estimated that this process may develop temperatures in a waste matter stream of up to 5,000 degrees Celsius, pressures of up to 1,000 atmospheres, and heating and cooling rates above 10 billion degrees Celsius per second for durations of less than one microsecond.

Apply ultrasonic energy to the waste matter in a batch process suffers from several drawbacks. Batch processing may be relatively slow and the efficiency with which ultrasonic energy is transmitted to waste matter contained in batch may be so low as to leave an unacceptable level of contaminants in the waste matter stream.

### SUMMARY OF THE INVENTION

The present invention is directed toward methods and apparatuses for treating a fluid mixture with ultrasonic energy. One such method includes introducing a flow of a mixture, such as an aqueous mixture, that includes a selected constituent, such as a contaminant, into a treatment apparatus including a treatment vessel. Ultrasonic energy is directed into the mixture as the mixture flows through the treatment vessel.

The invention is also directed toward a fluid waste matter treatment apparatus including a treatment vessel having an inlet and an outlet. The inlet receives a flow of the mixture into the treatment vessel and the outlet expels a flow of the mixture from the treatment vessel. An ultrasonic energy source is operatively coupled to the treatment vessel to transmit ultrasonic energy to the mixture at a selected energy level and selected frequency. In an alternate embodiment of the invention, two or more ultrasonic energy sources may be operatively coupled to the treatment vessel to transmit ultrasonic energy to the mixture at selected energy levels and selected frequencies. The fluid waste matter treatment apparatus may include one or more treatment vessels. Each treatment vessel may include a plurality of fluidly connected channels. In one embodiment, a first ultrasonic energy source is positioned to direct a first ultrasonic energy into the first channel and a second ultrasonic energy source positioned to direct second ultrasonic energy into the second channel. In another embodiment of the invention, an ultrasonic energy source is coupled to each of the plurality of fluidly connected channels.

Exposing a fluid mixture, such as a waste matter stream, to ultrasonic energy may cause chemical and/or physical changes to occur in the mixture. Temperatures and pressures developed by the collapsing cavitation bubbles may have several effects on the constituents of a waste matter stream. For example, the collapsing bubbles may form radicals, such as OH radicals which are unstable and may chemically interact with adjacent constituents in the waste matter stream to change the chemical composition of the adjacent constituents. In one such process, an OH radical reacts with nitrates in the waste matter stream to produce gases such as nitrogen dioxide. The following are sample steps in such a reaction:

[1] NO₃⁻+.OH_.NO₃+OH⁻

[2] .NO₃⁻+.OH_H₂O.+.NO₂

[3] .NO₂+.NO_{2 —}.NO+.NO₃

[4] .NO₂+.NO_{2—}.NO+.NO+O₂

[5].NO₂+.H_.NO+.OH

[6].NO₂+.OH_.NO+O₂.

[7].NO₂+.O._.NO₂+O₂

In another embodiment, the reaction may continue, for example, in the presence of additional constituents to produce nitrites. In yet another embodiment, the cavitating bubble may alter trichloroethylene, for example, in accordance with the following simplified reaction:

[1] (Cl)₂C═CHCl+2H₂O_ . . . _Cl₂+HCl+2H₂+2CO

In other embodiments, the collapsing cavitation bubbles may have effects on other molecules that change a chemical composition of the molecules and/or change a phase of the molecules from a liquid or solid phase to a gaseous phase.

In still further embodiments, the collapsing cavitation bubbles may have effects on other constituents of the waste matter stream. For example, the combination of increased pressure and cavitation bubbles may disrupt a molecular structure of an organism and accordingly kill pathogenic organisms, such as bacteria. Temperatures and pressures observed in the presence of collapsing cavitation bubbles may serve to alter structure of living cells and combust or oxidize constituents of the waste matter stream. For example, the high temperature produced by the collapsing cavitation bubble may oxidize constituents of the waste matter stream, producing by-products such as carbon dioxide and ash. The carbon dioxide may evolve from the waste matter stream and the ash may be filtered from the waste matter stream, as will be described in greater detail below. In still another embodiment, the collapsing cavitation bubbles may separate constituents of the waste matter stream. For example, when the waste matter stream includes a mixture of oil, water, and an emulsifier, the collapsing cavitation bubbles may alter the molecular characteristics of the emulsifier and cause the emulsifier to lose its effectiveness.

Accordingly, oil and water may separate from each other and one or the other may be removed from the stream. Collapsing cavitation bubbles may have other effects on the waste matter stream that alter the characteristics of the constituents of the stream in a manner that makes the constituents more benign and/or allows the constituents to be more easily removed from the waste matter stream. In an alternate aspect of the invention, a chemical composition including a selected constituent may be oxidized to produce an ash and a gas. The mixture may be contained under pressure while it is exposed to ultrasonic energy. The treatment vessel may be pneumatically coupled to a vacuum source after being exposed to the ultrasonic energy to remove gas from the mixture. In still a further aspect of the invention, the mixture may be exposed to a first ultrasonic energy having a first energy and a first frequency and the mixture may be exposed to a second ultrasonic energy having a second energy and a second frequency.

### BRIEF DESCRIPTION OF THE DRAWINGS

FIG. 1 is a schematic diagram of a fluid mixture treatment apparatus;

FIG. 2 is a schematic diagram of a waste matter treatment apparatus;

FIG. 3 is a partially schematic, isometric view of fluid mixture treatment assembly that forms a portion of the fluid mixture treatment apparatus shown in FIGS. 1 and 2;

FIG. 4 is a cutaway top view of a channel assembly that forms a portion of the waste matter treatment assembly shown in FIG. 3; and

FIGS. 5A-5C are schematic illustrations of alternate waste matter treatment assemblies.

### DETAILED DESCRIPTION

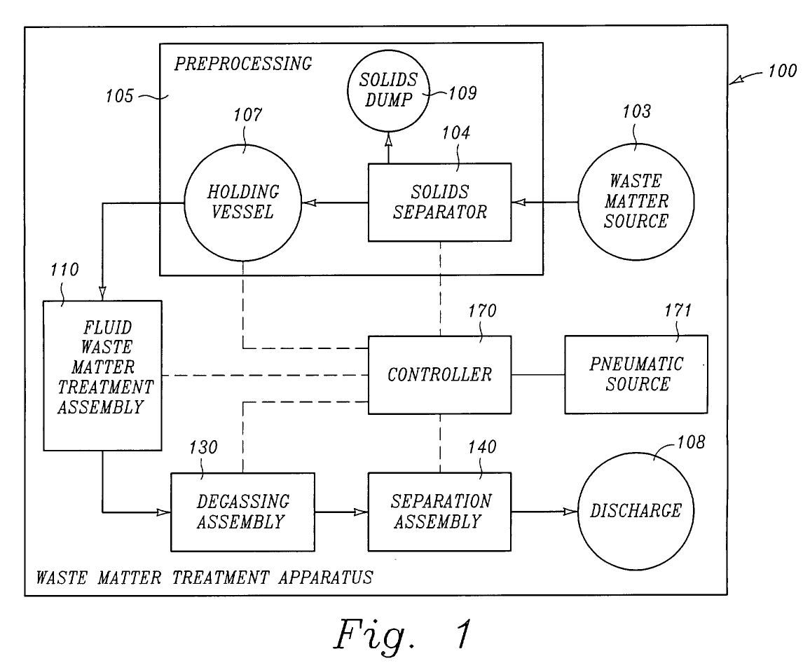

Specific details of certain embodiments of apparatuses and methods for treating a fluid mixture, such as an aqueous streams including waste matter, are set forth in the following description and in FIGS. 1-5. One skilled in the art, however, will understand that the present invention may have several additional embodiments, or that the invention may be practiced without several of the details described below.

Referring to FIG. 1, a schematic diagram of fluid mixture treatment apparatus 100 is shown including preprocessing assembly 105. Preprocessing assembly 105 includes waste matter source 103, solids separator 104, holding vessel 107 and solids dump 109. Waste matter flow may proceed from solids separator 104 to holding vessel 107 fluidly coupled to fluid mixture treatment assembly 110. Fluid mixture treatment apparatus 100 may also include solids separator 104, shown located between waste matter source 103 and fluid mixture treatment assembly 110. Solids separator 104 may remove a selected quantity of solids suspended in the waste matter and direct the removed solids to solids dump 109. Fluid mixture treatment assembly 110 is also fluidly coupled to degassing assembly 130 where gaseous constituents are removed from the flow. Fluid mixture treatment apparatus 100 may also include separation assembly 140 where solid components which may have been generated in fluid mixture treatment assembly 110 are removed from the flow. Processed fluid is discharged from separation assembly 140 at discharge 108. Fluid mixture treatment apparatus 100 may be automatically controlled by controller 170. Controller 170 is operatively coupled to a pneumatic source 171 to direct and regulate flows of pressurized air to pneumatically controlled elements of fluid mixture treatment apparatus 100.

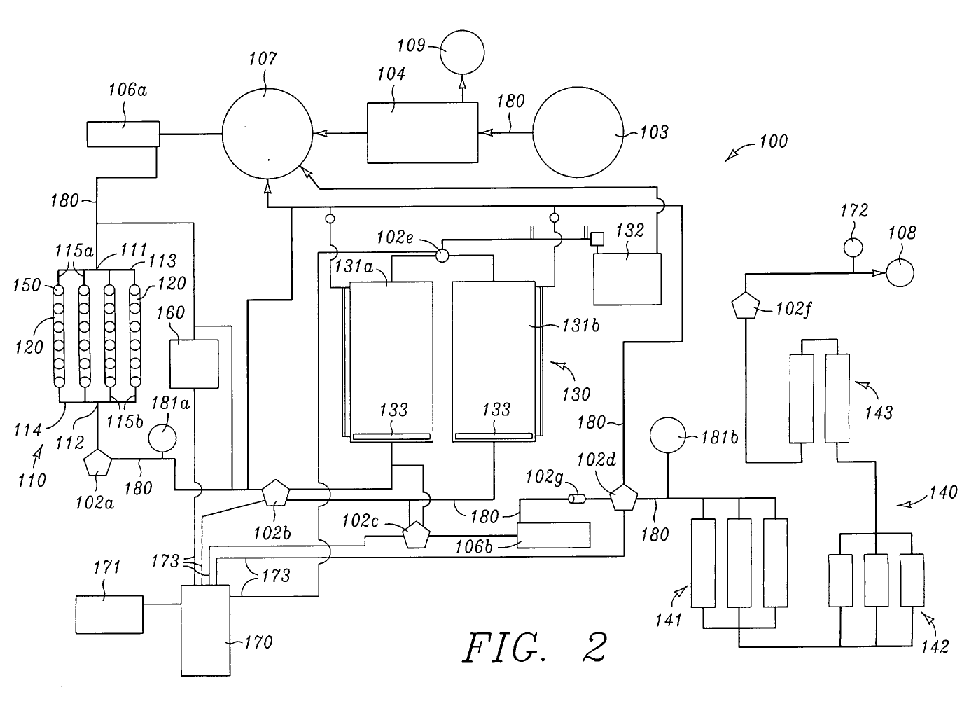

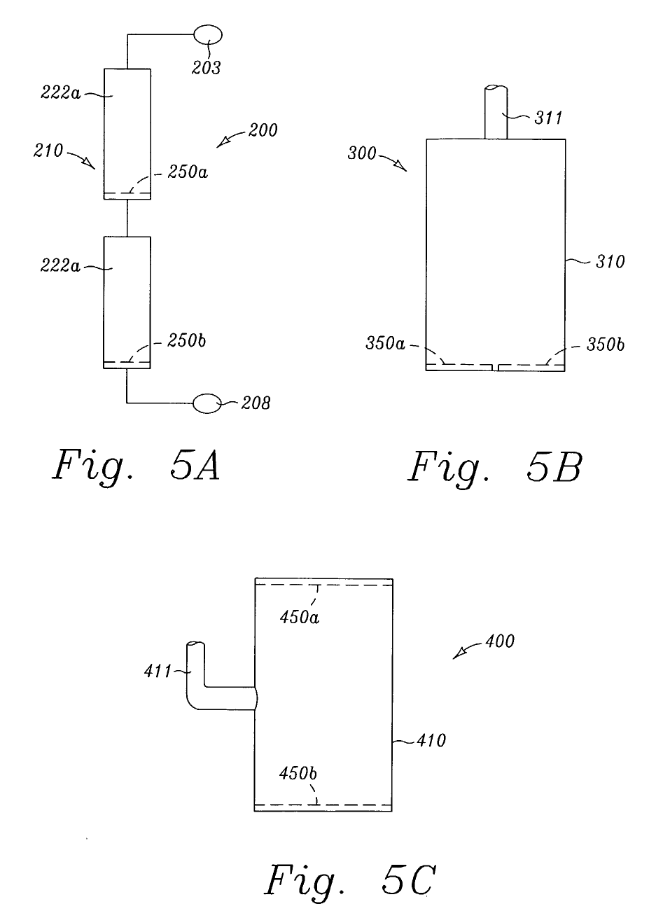

Referring to FIG. 2, fluid mixture treatment apparatus 100 will be described in further detail. Waste matter source 103 is fluidly coupled by process piping 180 to fluid mixture treatment assembly 110. Fluid mixture treatment assembly 110 may include one or more channel assemblies 120 having ultrasonic energy sources 150 that direct ultrasonic energy into the waste matter flow to gasify and/or alter a chemical structure of constituents in the flow. The flow proceeds from fluid mixture treatment assembly 110 via process piping 180 to degassing assembly 130 where gaseous components are removed from the flow. The degassed stream then proceeds to a separation assembly 140 where solid components which may have been generated in fluid mixture treatment assembly 110 are removed from the flow. The flow exits fluid mixture treatment apparatus 100 through discharge 108 and may then be reused or returned to the environment. In one aspect of this embodiment, the waste matter stream proceeds in a continuous manner from the waste matter source 103 to the discharge 108. Alternatively, fluid mixture treatment apparatus 100 may operate in a batch mode, as will be described in greater detail below with reference to FIGS. 5B-5C.

In one embodiment, fluid mixture treatment apparatus 100 also includes solids separator 104, shown located between waste matter source 103 and fluid mixture treatment assembly 110. Solids separator 104 may remove a selected quantity of solids suspended in the waste matter and direct the removed solids to a solids dump 109. Removing at least a portion of the solids from the waste matter stream at preprocessing assembly 105 upstream of fluid mixture treatment assembly 110 may improve the efficiency with which fluid mixture treatment apparatus 100 operates, as will be described in greater detail below.

The waste matter flow may proceed from solids separator 104 to holding vessel 107. Pump 106a withdraws waste matter from holding vessel 107, pressurizes the waste matter, and directs the waste matter to fluid mixture treatment assembly 110 via process piping 180. In one embodiment, the pressure of waste matter entering fluid mixture treatment assembly 110 may be from about 5 psi to about 40 psi, but in other embodiments the pressure of waste matter entering fluid mixture treatment assembly 110 may be outside of this range.

In one embodiment, fluid mixture treatment assembly 110 may include inlet 111 that receives a continuous flow of waste matter and outlet 112 through which a continuous flow of treated fluid mixture exits fluid mixture treatment assembly 110. In one aspect of this embodiment, fluid mixture treatment assembly 110 may be configured to divide the waste matter stream into several components that are processed in parallel in the channel assemblies 120 and recombined before exiting fluid mixture treatment assembly 110 through outlet 112. Accordingly, fluid mixture treatment assembly 110 may include intake manifold 113 for dividing incoming flow upstream of channel assemblies 120 and output manifold 114 for collecting the flow downstream of channel assemblies 120. Intake manifold 113 may further include a plurality of assembly intakes 115a, each of which directs a portion of the incoming waste matter into one of the channel assemblies 120. Assembly outlets 115b collect flows from the channel assemblies 120 upstream of outlet 112.

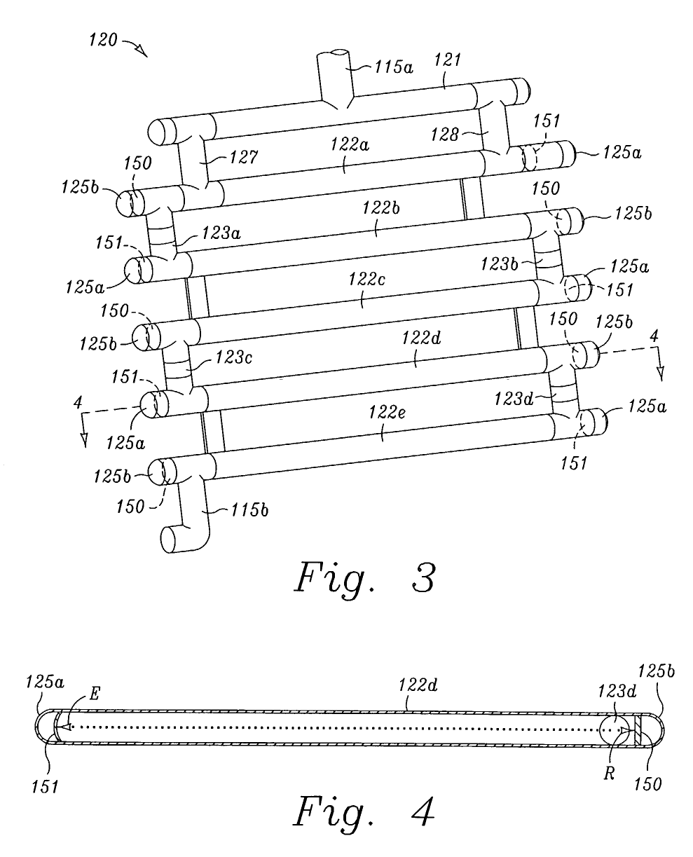

FIG. 3 is an isometric view of a single channel assembly 120. In the embodiment shown, channel assembly 120 includes inlet manifold 121 that directs waste matter flow from assembly intake 115a into a plurality of channels 122a-122e which are serially and hydraulically connected by conduits 123a-123d. In the embodiment shown, each channel 122 is configured as a pipe having a first end 125a and a second end 125b. Each of the plurality of channels 122a-122e may be supported relative to adjacent channels by a plurality of braces 124. The waste matter stream proceeds generally from first end 125a of each of the plurality of channels 122a-122e to the second end 125b, then through conduit 123a-123d to the first end 125a of the next of the plurality of channels 122a-122e. The waste matter stream passes from the last serially and hydraulically connected channel 122e into assembly outlet 115b, and then to outlet port 112 of fluid mixture treatment assembly 110, shown in FIG. 2.

In the embodiment shown in FIG. 3, ultrasonic energy source 150, such as a piezoelectric source or another ultrasonic energy emitter or generator, is positioned within each of the plurality of channels 122a-122e in or near second end 125b. Accordingly, the waste matter may flow toward ultrasonic energy source 150 as it moves through the plurality of channels 122a-122e. Alternatively, ultrasonic energy source 150 may be positioned toward first end 125a of the plurality of channels 122a-122e with the flow of waste matter flowing away from ultrasonic energy source 150. In either embodiment, the end of each of the plurality of channels 122a-122e, opposite ultrasonic energy source 150 may include a reflector 151 positioned to reflect at least a portion of the ultrasonic energy generated by ultrasonic energy source 150. Accordingly, reflector 151 may direct ultrasonic energy and/or the products produced by the ultrasonic energy back toward the ultrasonic energy source 150. Whether or not channel assembly 120 includes reflectors 151, ultrasonic energy source 150 may be selected to emit ultrasonic energy at a selected energy level and a selected frequency that causes a liquid or aqueous portion of the waste matter stream to cavitate.

Referring to FIG. 4, a cutaway top view of a channel 122d is shown to advantage. Ultrasonic energy source 150 is positioned within channel 122d near second end 125b. Reflector 151 is positioned within channel 122d near first end 125a. As waste matter flows towards ultrasonic energy source 150 ultrasonic energy source 150 transmits ultrasonic energy through waste matter and towards first end 125a as indicated by the arrow E. At least a portion of the ultrasonic energy is reflected through the waste matter and towards second end 125b as indicated by the arrow R.

Characteristics of both channel assembly 120 and ultrasonic energy source 150 may be selected to have desired effects on the waste matter stream. For example, the frequency of the ultrasonic energy transmitted by the ultrasonic energy source 150 into the waste matter stream may be selected based on the resonant frequencies of constituents in the waste matter stream. In one particular embodiment, the frequency of ultrasonic energy source 150 may be selected to be at or above a natural resonant frequency of molecules of constituents in the stream. In one further specific example, when the flow includes farm animal fecal waste in an aqueous solution, along with pathogens such as e-coli, ultrasonic energy source 150 may be selected to produce a distribution of ultrasonic waves having an energy peak at approximately 980 kilohertz. In other embodiments, the peak energy of the ultrasonic energy sources 150 may be selected to occur at other frequencies, depending for example on the types, relative quantities, and/or relative potential harmful effects of constituents in the stream. Accordingly, individual ultrasonic energy sources 150 may be selected to have a particular, and potentially unique, effect on selected constituents of the waste matter stream.

In another embodiment, adjacent ultrasonic energy sources within one or more channel assemblies 120 may produce different frequencies. For example, the ultrasonic energy source 150 in the uppermost channel 122a of FIG. 3 may emit energy at a higher frequency than that emitted by ultrasonic energy source 150 in the next downstream channel 122b.

An advantage of this arrangement for waste matter streams having multiple constituents, each of which is best affected by ultrasonic energy at a different frequency, is that the waste matter streams may be subjected to a plurality of ultrasonic energy sources each having selected frequencies and energy levels, with each frequency and energy level selected to affect a particular constituent of the waste matter stream. Such an arrangement may be more effective than some conventional arrangements for removing constituents from the waste matter stream in a single apparatus.

The geometry of channel assembly 120 may be selected to define the time during which any given constituent of the waste matter stream is subjected to the energy emitted by the ultrasonic energy sources 150. For example, the overall length of the flow path through each channel assembly 120 and the rate at which the waste matter stream passes through the channel assembly 120 may be selected according to the amount of suspended solids in the waste matter stream, with the overall residence time within the channel assembly 120 being lower for waste matter streams having relatively few suspended solids and higher for waste matter streams having more suspended solids. Accordingly, each channel assembly 120 may be made smaller by reducing the number of channels 122a-122e in each channel assembly 120 and/or faster by increasing the flow rate of the waste matter through the channel assembly 120 when solids separator 104, shown in FIG. 2, filters out a greater fraction of the suspended solids.

Referring again to FIG. 2, fluid mixture treatment apparatus 100 may include features that increase the number of radicals and/or other chemically reactive constituents in the waste matter stream. For example, the apparatus may include an ozone generator 160 fluidly coupled to fluid waste matter treatment assembly 110 to introduce ozone into the waste matter stream while the ultrasonic energy sources 150 are energized.

In other embodiments, the ozone generator 160 may be replaced with, or supplemented by, sources of other chemically reactive species. In any of these embodiments, gas generated by the chemical reactions in fluid waste matter treatment assembly 110 may be removed from the waste matter stream, as will be described in greater detail below. The non-gas molecules remaining in the waste matter stream after the gas is formed may either be removed from the waste matter stream or may remain in the waste matter stream depending, for example, on the potential hazard to the quality of the waste matter presented by the remaining molecules.

In one embodiment, the waste matter stream may proceed from fluid waste matter treatment assembly 110 toward the degassing assembly 130 via the process piping 180. In one aspect of this embodiment, fluid mixture treatment apparatus 100 may include a valve 102a, such as a throttling valve, that allows the portion of the waste matter stream upstream of valve 102a to have a pressure greater than atmospheric pressure, while the portion of the waste matter stream downstream of the valve 102a may be subjected to a pressure less than atmospheric pressure. Accordingly, the pressure within degassing assembly 130 may be reduced to increase the rate at which gas evolves from the mixture, without reducing the pressure of the mixture within fluid waste matter treatment assembly 110.

Degassing assembly 130 may include two gas release chambers shown as a first chamber 131a and a second chamber 131b hydraulically connected to process piping 180 with a selector valve 102b. Selector valve 102b may be configured to alternate between a first setting with the waste matter stream directed into the first gas release chamber 131a and a second setting with the waste matter stream directed into the second gas release chamber 131b. The waste matter stream exiting fluid waste matter treatment assembly 110 may accordingly be directed into the first gas release chamber 131a until first chamber 131a is filled to a desired level, and then directed in the second gas release chamber 131b.

While the second gas release chamber 131b is filling, the filled first gas release chamber 113a may be subjected to a vacuum pressure generated by a vacuum source 132 fluidly coupled to gas release chambers 131a and 131b with valve 102e. After the waste matter has resided in the first gas release chamber 131a under vacuum for a time sufficient to remove a selected amount of gas from the waste matter stream, the stream exits the first chamber 131a and first chamber 131a is re-filled while a vacuum is applied to the waste matter in second chamber 131b. Accordingly, the continuous flow of waste matter from fluid waste matter treatment assembly 110 may be sequentially directed into either the first or second gas release chamber 131a or 131b without interrupting flow. In one embodiment, vacuum source 132 may remain in fluid communication with both gas release chambers 131a and 131b during both the transient “fill” and the steady state “filled” portions of the cycle for each chamber. Alternatively, vacuum source 132 may be fluidly coupled to each gas release chamber 131a and 131b only after that gas release chamber 131a or 131b has been filled. In either embodiment, vacuum source 132 may increase the speed with which gas in the waste matter is removed.

In an alternate embodiment, gas release chambers 131 may be open to the atmosphere to release gas from the waste matter stream under atmosphere pressure Whether the waste matter is subject to atmospheric pressure or less than atmospheric pressure, the fluid within chambers 131a and 131b may be agitated, for example, with agitation device 133. In one aspect of this embodiment, agitation device 133 may include a piezoelectric energy source that generates ultrasonic energy in the gas release chambers 131a and 131b. Alternatively, agitation device 133 may generate pressure waves at other frequencies. In other embodiments, agitation device 133 may include other devices, such as strainers or other mechanical implements.

After exiting the degassing assembly 130, the waste matter stream proceeds to the separation assembly 140 via process piping 180. Valve 102c may be selectively adjusted to drain flow from whichever gas release chamber 131a or 131b has completed its cycle. Pump 106b pressurizes the waste matter stream to direct the stream through a check valve 102g and into first, second and third filter stages 141, 142 and 143 in separation assembly 140. In one embodiment, first filter stage 141 includes multi-media micron filter elements, the second filter stage 142 may include two micron filter elements and third filter stage 143 may include activated charcoal. In another embodiment, separation assembly 140 may include other separation arrangements. Back pressure valve 102f controls back pressure through separation assembly 140, and flow meter 172 monitors a rate of flow through fluid mixture treatment apparatus 100. When flow meter 172 is positioned adjacent to the discharge 108, as shown in FIG. 2, the flow rate determined by flow meter 172 may be less than a flow rate measured at waste matter source 103 because gas may be removed from the flow at degassing assembly 130 and solids may be removed from the flow in the separation assembly 140.

In one embodiment, the operation of fluid mixture treatment apparatus 100 may be automatically controlled by controller 170. In one aspect of this embodiment, controller 170 is operatively coupled to a pneumatic source 171 to direct and regulate flows of pressurized air to the controlled elements via pneumatic lines 173. Fluid mixture treatment apparatus 100 may include other automatic control features, such as failure sensing devices in valves 102b and 102d that close these valves automatically in the event of a power failure to direct the waste matter stream back to the waste matter holding vessel 107. Surge suppression tanks 181a and 181b may be positioned along the flow path between the waste matter source 103 and the discharge 108 to absorb fluctuations in the flow volume and pressure throughout fluid mixture treatment apparatus 100.

One feature of an embodiment of fluid mixture treatment apparatus 100 described above with reference to FIGS. 2 and 3 is that the waste matter stream flows in a continuous fashion from waste matter source 103 to outlet 108.

An advantage of this feature is that the treatment of the waste matter throughout fluid mixture treatment apparatus 100 may be more consistent and faster than conventional batch systems. Another feature of an embodiment of fluid mixture treatment apparatus 100 is that the channel assemblies 120 may have a modular construction. Accordingly, channel assemblies 120 may be configured having as long or as short a flow path as is appropriate for the type of flow directed into the assemblies.

Still a further advantage is that fluid waste matter treatment assembly 110 may include channel assemblies 120 having different flow path lengths. For example, each channel assembly 120 may have a different flow path length, and instead of directing equal portions of the waste matter stream through each channel assembly 120, the entire waste matter stream may be directed through the channel assembly 120 having the length corresponding to the desired residence time appropriate for the amount of solids suspended in that waste matter stream. Accordingly, an embodiment of fluid mixture treatment apparatus 100 may be suitable for treating a variety of different waste matter streams. Still another feature of the embodiment of fluid mixture treatment apparatus 100 described above with reference to FIGS. 2 and 3 is that fluid waste matter treatment assembly 110 may include a plurality of ultrasonic energy sources 150, each emitting ultrasonic energy at a different frequency. Accordingly, each ultrasonic energy source 150 may be selected to have a desired effect on a particular constituent of the waste matter.

In one aspect of this embodiment, a plurality of ultrasonic energy sources 150 having different frequencies may be disposed in each channel assembly 120. Alternatively, all the ultrasonic energy sources 150 in a particular channel assembly 120 may emit ultrasonic energy at the same frequency, but the frequency selected for each channel assembly 120 may be different. Accordingly, fluid mixture treatment apparatus 100 may be compatible with a variety of different waste matter streams by directing a selected waste matter stream through the channel assembly 120 having ultrasonic energy sources 150 that emit energy at the frequency most appropriate for the constituents in that waste matter stream.

FIGS. 5A-5C are schematic illustrations of portions of treatment apparatuses in accordance with other embodiments of the invention. For purposes of illustration, only portions of the apparatuses are shown in FIGS. 5A-5C, and it will be understood that the apparatuses may include additional elements that are generally similar to those described above with reference to FIGS. 2 and 3.

FIG. 5A illustrates a portion of fluid mixture treatment vessel 200 that includes a waste matter source 203, an outflow port 208 and treatment vessel 210 between the source 203 and the outflow port 208. In one aspect of this embodiment, treatment vessel 210 may include two channels 222, (shown as first channel 222a and a second channel 222b), hydraulically connected together in a series arrangement. First channel 222a includes first ultrasonic energy source 250a that emits ultrasonic energy at a first frequency, and second channel 222b may include second ultrasonic energy source 250b that emits ultrasonic energy at a second frequency. Accordingly, fluid mixture treatment vessel 200 may direct ultrasonic energy at different frequencies into the same waste matter stream to selectively affect different constituents within the waste matter stream, as described above with reference to FIGS. 2-3. Alternatively, first and second energy sources 250a and 250b may emit ultrasonic energy at the same frequency. In either embodiment, each channel 222 can include a single length of a tube, a series of channel segments that double back on each other, similar to those shown in FIG. 3, a non-tubular chamber, or any liquid-tight container.

FIG. 5B illustrates fluid mixture treatment vessel 300 that operates in a batch mode and includes treatment vessel 310 having port 311 which serves both as an inlet and an outlet for the waste matter to be treated. Fluid mixture treatment vessel 300 also includes first and second ultrasonic energy sources 350a and 350b. As was generally described above with reference to FIGS. 1-5A, the first ultrasonic energy source 350a may emit ultrasonic energy at a first frequency, and the second ultrasonic energy source 350b may emit ultrasonic energy at a second frequency different than the first frequency. Ultrasonic energy sources 350a and 350b may be placed at any position within treatment vessel 310 for which the ultrasonic energy may be efficiently transmitted to the waste matter stream. For example, both ultrasonic energy sources 350a and 350b may be positioned at one end of treatment vessel 310 and, in one embodiment, an ultrasonic reflector (not shown) may be positioned at the opposite end. In any of the embodiments described above with reference to FIG. 5B, one feature of fluid mixture treatment vessel 300 is that it can be used in situations where a batch operation is preferred to a continuous flow operation.

FIG. 5C illustrates an fluid mixture treatment vessel 400 including treatment vessel 410 with inlet 411 and two ultrasonic energy sources, first source 450a and second source 450b at opposite ends of treatment vessel 410. Accordingly, the energy sources 450 may be operated either simultaneously or sequentially to create cavitation bubbles in a volume of waste matter within treatment vessel 410. In one aspect of this embodiment, each of the energy sources 450 may be configured and positioned to reduce potential wear caused by energy emitted by the other energy source 450.

From the foregoing it will be appreciated that specific embodiments of the invention have been described herein for purposes of illustration, but that various modifications may be made without deviating from the spirit and scope of the invention. For example, several embodiments of the invention have been described in the context of an aqueous mixture or waste matter stream, and in other embodiments, the mixture may not include water. In still another embodiment, the mixture may include a gaseous component. In still another embodiment, the apparatus may include first and second treatment vessels and may receive a continuous flow of waste matter that is alternately directed into each treatment vessel. The first treatment vessel may be filled first, after which the continuous flow is directed into the second treatment vessel. While the second treatment vessel is filling, ultrasonic energy may be directed into the mixture in the first treatment vessel. Alternately, while the first treatment vessel is filling, ultrasonic energy may be directed into the mixture in the second treatment vessel. Accordingly, the apparatus may take in a continuous flow of waste matter that is divided and exposed to ultrasonic energy in separate batch processes. Accordingly, the invention is not limited except as by the appended claims. Various modifications to the described embodiments as well as the inclusion or exclusion of additional embodiments will be apparent to persons skilled in the art upon reference to this description. It is therefore contemplated that the appended claims will cover any such modifications or embodiments as fall within the true scope of the invention

I claim:

1. A method for treating a fluid mixture with ultrasonic energy including the steps of:
directing a flow of the fluid mixture including a selected constituent into a treatment apparatus; and
transmitting ultrasonic energy into the fluid mixture as the fluid mixture flows through the treatment apparatus to alter a molecular composition of at least one constituent and generate a gas.

2. The method of claim 1 further comprising:
directing the fluid mixture flow into a first channel;
transmitting ultrasonic energy to the fluid mixture flow from a first ultrasonic energy generator while the fluid mixture flow passes through the first channel;
directing the fluid mixture flow into a second channel directly from the first channel; and
transmitting ultrasonic energy to the fluid mixture flow from a second ultrasonic energy generator while the fluid mixture flow passes through the second channel.

3. The method of claim 1 further comprising:
directing the fluid mixture flow into a first channel;
transmitting ultrasonic energy to the fluid mixture flow from a first ultrasonic energy generator at a first frequency while the fluid mixture flow passes through the first channel;
directing the fluid mixture flow into a second channel; and
transmitting ultrasonic energy to the fluid mixture flow from a second ultrasonic energy generator at a second frequency different than the first frequency while the fluid mixture flow passes through the second channel.

4. The method of claim 1 wherein directing ultrasonic energy through the flow includes cavitating a liquid portion of the fluid mixture to generate heat, and wherein altering a chemical composition of the selected constituent includes oxidizing the selected constituent to produce an ash and a gas.

5. The method of claim 1 wherein directing ultrasonic energy through the flow includes cavitating a liquid portion of the fluid mixture to generate heat, and wherein the method further includes killing pathogens in the fluid mixture by exposing the pathogens to the heat.

6. The method of claim 1 wherein directing ultrasonic energy through the flow includes cavitating a liquid portion of the fluid mixture.

7. The method of claim 1 wherein the fluid mixture flow initially includes suspended solids, and wherein the method further comprises removing at least a portion of the suspended solids before exposing the fluid mixture flow to ultrasonic energy.

8. The method of claim 1 further comprising:
directing the fluid mixture flow into a first channel;
transmitting ultrasonic energy to the fluid mixture flow from a first ultrasonic energy generator while the fluid mixture flow passes through the first channel;
redirecting the fluid mixture at least approximately 180 degrees into a second channel; and
transmitting ultrasonic energy to the fluid mixture flow from a second ultrasonic energy generator while the fluid mixture flow passes through the second channel.

9. The method of claim 1 further comprising directing the fluid mixture flow into a plurality of channels having a length corresponding to a level of suspended solids in the fluid mixture.

10. The method of claim 1 further comprising removing at least a portion of the selected constituent from the fluid mixture flow after exposing the selected constituent to the ultrasonic energy and while the fluid mixture flow passes continuously through the apparatus.

11. The method of claim 1 wherein the ultrasonic energy includes a first ultrasonic energy having a first frequency, and wherein the method further comprises exposing the selected constituent to a second ultrasonic energy having a second frequency different than the first frequency.

12. The method of claim 1 wherein the ultrasonic energy includes a first ultrasonic energy having a first frequency, and wherein the method further comprises exposing the selected constituent to a second ultrasonic energy having a second frequency different than the first frequency after exposing the selected constituent to the first frequency.

13. The method of claim 1 further comprising dividing the flow into first and second portions, conveying the first and second portions along separate flow paths and exposing the selected constituent in the first portion of the flow to ultrasonic energy while simultaneously exposing the selected constituent in the second portion of the flow to ultrasonic energy.

14. The method of claim 1 further comprising pressurizing the fluid mixture and altering the phase and/or chemical composition of the selected constituent while the fluid mixture flow is pressurized.

15. The method of claim 1 wherein a molecular structure of a component of the fluid mixture flow has a resonant frequency, and wherein the method further comprises selecting a frequency of the ultrasonic energy to be at or above the resonant frequency of the fluid mixture flow.

16. The method of claim 1 wherein a molecular structure of a component of the fluid mixture flow has a resonant frequency, and wherein the method further comprises selecting an ultrasonic energy frequency based upon the molecular structure of a component.

17. The method of claim 1 wherein the fluid mixture flow includes water, and wherein the method further includes separating an OH radical from a molecule of the water and combining the OH radical with a molecule of the selected constituent.

18. The method of claim 1 wherein altering a phase of the selected constituent includes changing the phase of the selected constituent from a solid to a gas.

19. The method of claim 1 further comprising introducing an oxygen radical into the fluid mixture flow before exposing the selected constituent to the ultrasonic energy.

20. The method of claim 1 further comprising introducing ozone into the fluid mixture flow before exposing the selected constituent to ultrasonic energy.

21. The method of claim 1 wherein the selected constituent includes an emulsifier and further wherein altering a phase and/or chemical composition of the selected constituent includes deactivating the emulsifier.

22. A method for removing contaminants from water, comprising:
introducing a flow of a fluid mixture of the water and the contaminants into a treatment apparatus;
introducing ultrasonic energy into the fluid mixture as the fluid mixture flows through the treatment apparatus to alter a molecular composition of at least one of the contaminants and generate a gas; and
applying a vacuum to the fluid mixture to remove at least some of the gas from the fluid mixture as the fluid mixture flows through the treatment apparatus.

23. The method of claim 22 wherein the ultrasonic energy is a first ultrasonic energy and wherein the method further comprises introducing a second ultrasonic energy to the fluid mixture.

24. The method of claim 22 further comprising filtering solid materials from the fluid mixture after applying a vacuum to the fluid mixture.

25. The method of claim 22 further comprising pressurizing the fluid mixture and introducing ultrasonic energy into the fluid mixture while the fluid mixture flows under pressure.

26. The method of claim 22 further comprising:
directing the fluid mixture into a first channel;
transmitting ultrasonic energy to the fluid mixture from a first ultrasonic energy generator while the fluid mixture flows through the first channel;
directing the fluid mixture into a second channel; and
transmitting ultrasonic energy to the fluid mixture from a second ultrasonic energy generator while the fluid mixture flows through the second channel.

27. The method of claim 22 further comprising directing the fluid mixture into a channel having a length corresponding to a level of suspended solids in the fluid mixture.

28. The method of claim 22 further comprising removing at least a portion of the contaminants from the fluid mixture after exposing the contaminants to the ultrasonic energy and while the fluid mixture flows continuously through the apparatus.

29. The method of claim 22 wherein the ultrasonic energy includes a first ultrasonic energy having a first frequency, and wherein the method further comprises exposing the contaminants to a second ultrasonic energy having a second frequency different than the first frequency.

30. The method of claim 22 wherein the ultrasonic energy includes a first ultrasonic energy having a first frequency, and wherein the method further comprises exposing the contaminants to a second ultrasonic energy having a second frequency different than the first frequency after exposing the contaminants to the first frequency.

31. The method of claim 22 wherein a molecular structure of a contaminant has a resonant frequency, and wherein the method further comprises selecting a frequency of the ultrasonic energy to be at or above the resonant frequency of the contaminant.

32. The method of claim 22 further comprising introducing ozone into the fluid mixture before exposing the contaminants to ultrasonic energy.

33. A method for removing a contaminant from a fluid mixture including the contaminant, the method including the steps of:
introducing a flow of a fluid mixture including water and the contaminant into a fluid waste matter treatment apparatus;
pressurizing the fluid waste matter treatment apparatus;
applying ultrasonic energy into the fluid mixture as the fluid mixture flows through the fluid waste matter treatment apparatus under pressure causing transient cavitation in the fluid mixture disrupting a molecular structure of the contaminant;
producing a gas from chemical interactions between the contaminant and at least one other constituent of the water; and
applying a vacuum to the fluid mixture to remove at least some of the gas from the fluid mixture as the fluid mixture flows through treatment apparatus.

34. The method of claim 33 wherein introducing the ultrasonic energy includes introducing a first ultrasonic energy having a first frequency and introducing a second ultrasonic energy having a second frequency different than the first frequency.

35. The method of claim 33 wherein the contaminants include farm animal fecal waste, and wherein introducing ultrasonic energy includes directing ultrasonic energy having a frequency of 980 kilohertz.

36. A method for treating a selected constituent in a fluid mixture that includes the selected constituent, the method comprising:
introducing the fluid mixture into a treatment apparatus;
directing a first ultrasonic energy including a first frequency into the fluid mixture to alter a phase and/or a chemical composition of the selected constituent; and
directing a second ultrasonic energy at a second frequency into the fluid mixture, the second frequency being different than the first frequency.

37. The method of claim 36 wherein the apparatus includes a first ultrasonic emitter directing the first ultrasonic energy into the fluid mixture and a second ultrasonic emitter directing the second ultrasonic energy into the fluid mixture, the second ultrasonic emitter being spaced apart from the first ultrasonic emitter, and wherein the method further comprises:
directing the fluid mixture from the first ultrasonic emitter to the second ultrasonic emitter; and
exposing a portion of the fluid mixture to the second ultrasonic energy after exposing the portion of the fluid mixture to the first ultrasonic energy.

38. The method of claim 36 further comprising exposing a portion of the fluid mixture to the first ultrasonic energy simultaneously with exposing the portion of the fluid mixture to the second ultrasonic energy.

39. The method of claim 36 wherein the first and second ultrasonic energies are directed into the fluid mixture while the fluid mixture flows continuously through the apparatus.

40. The method of claim 36 further comprising removing at least a portion of the selected constituent from the fluid mixture after exposing the selected constituent to the first and second ultrasonic energies and while the fluid mixture flows continuously through the apparatus.

41. The method of claim 36 wherein the selected constituent is a first selected constituent having a first molecular structure and the fluid mixture includes a second selected constituent having a second molecular structure different than the first molecular structure, and wherein the method further comprises altering a phase and/or a chemical composition of the second selected constituent with the second ultrasonic energy.

42. The method of claim 36 further comprising:
directing the fluid mixture into a first channel;
transmitting ultrasonic energy to the fluid mixture at the first frequency while the fluid mixture flows through the first channel;
directing the fluid mixture into a second channel; and
transmitting ultrasonic energy to the fluid mixture at the second frequency while the fluid mixture flows through the second channel.

43. The method of claim 36 further comprising directing the fluid mixture into a channel having a length corresponding to an amount of solid material suspended in the fluid mixture.

44. The method of claim 36 further comprising pressurizing the fluid mixture and altering the phase and/or a chemical composition of the selected constituent while the fluid mixture is pressurized.

45. A method for heating a selected constituent in a fluid mixture that includes the selected constituent, the method comprising:
introducing the fluid mixture into a treatment apparatus;
pressurizing the fluid mixture within the treatment apparatus; and
altering a phase and/or a chemical composition of the selected constituent by exposing the fluid mixture to ultrasonic energy while the fluid mixture is under pressure.

46. The method of claim 45 wherein the fluid mixture includes a liquid and exposing the fluid mixture to ultrasonic energy includes cavitating a portion of the liquid in the fluid mixture while the fluid mixture is under a pressure of from about 5 psi to about 40 psi.

47. The method of claim 45 wherein introducing the fluid mixture includes providing a first continuous flow of the fluid mixture through an inlet of the apparatus, and wherein the method further includes withdrawing a second continuous flow of the fluid mixture through an outlet of the apparatus and exposing the fluid mixture to the ultrasonic energy while the fluid mixture flows from the inlet to the outlet.

48. The method of claim 45 wherein the ultrasonic energy is a first ultrasonic energy having a first frequency and wherein the method further includes exposing the selected constituent to a second ultrasonic energy having a second frequency different than the first frequency.

49. The method of claim 45 wherein the fluid mixture includes water and wherein the method further comprises:
generating a gas by a chemical interaction between the selected constituent and constituents of water while the fluid mixture is under pressure; and
removing the gas from the fluid mixture by reducing the pressure to which the fluid mixture is subjected.

50. A method for treating a selected constituent in a fluid mixture that includes the selected constituent, the method comprising:
directing a flow of the fluid mixture with the selected constituent into a treatment apparatus; and
altering a phase and/or a chemical composition of the selected constituent by directing ultrasonic energy through the flow of the fluid mixture within the apparatus.

51. A fluid mixture treatment apparatus for treating a selected constituent in a fluid mixture, the apparatus comprising:
a treatment vessel including an inlet and an outlet, the inlet configured to receive a flow of the fluid mixture during operation and the outlet configured to expel the flow of the fluid mixture; and
an ultrasonic energy source operatively coupled to the treatment vessel, the ultrasonic energy source configured to transmit ultrasonic energy to the fluid mixture.

52. The apparatus of claim 51 wherein the source of ultrasonic energy further comprises a first source configured to emit ultrasonic energy at a first frequency and wherein the apparatus further comprises a second source of ultrasonic energy operatively coupled to the treatment vessel to transmit ultrasonic energy to the fluid mixture at a second frequency.

53. The apparatus of claim 51 wherein the selected constituent of the fluid mixture is a first selected constituent and the fluid mixture includes a second selected constituent, and further wherein the source of ultrasonic energy is selected to gasify and/or alter a chemical composition of the first selected constituent.

54. The apparatus of claim 51 wherein the selected constituent of the fluid mixture is a first selected constituent and the fluid mixture includes a second selected constituent, and further wherein the second source of ultrasonic energy is selected to gasify and/or alter a chemical composition of the second selected constituent.

55. The apparatus of claim 51 further comprising a pressure source in fluid communication with the treatment vessel to pressurize the fluid mixture as the fluid mixture moves from the inlet to the outlet.

56. The apparatus of claim 51 further comprising:
a degassing chamber coupled to the outlet of the treatment vessel;
a vacuum source coupled to the degassing chamber to draw gas from the fluid mixture; and
a valve between the degassing chamber and the treatment vessel to maintain a first pressure in the treatment vessel higher than a second pressure in the degassing chamber.

57. The apparatus of claim 51 wherein the source of ultrasonic energy is a first source and wherein the apparatus further comprises:
a degassing chamber coupled to the outlet of the treatment vessel; and
a second source of ultrasonic energy operatively coupled to the degassing chamber to remove gas from the fluid mixture.

58. The apparatus of claim 51 further comprising at least one filter in fluid communication with the outlet of the treatment vessel to separate solid material from the fluid mixture after the fluid mixture exits the treatment vessel.

59. The apparatus of claim 51 wherein the source of ultrasonic energy is a first source and wherein the treatment vessel includes a first channel and a second channel coupled to the first channel, the first source being positioned to direct a first ultrasonic energy into the fluid mixture as the fluid mixture passes through the first channel, and wherein the apparatus further comprises a second source of ultrasonic energy positioned to direct a second ultrasonic energy into the fluid mixture as the fluid mixture passes through the second channel.

60. The apparatus of claim 51 wherein the source of ultrasonic energy is a first source and wherein the treatment vessel includes a first channel and a second channel hydraulically connected to the first channel, the first source being positioned to direct a first ultrasonic energy at a first frequency into the fluid mixture as the fluid mixture passes through the first channel, and wherein the apparatus further comprises a second source of ultrasonic energy positioned to direct a second ultrasonic energy at a second frequency into the fluid mixture as the fluid mixture passes through the second channel.

61. The apparatus of claim 51 wherein the fluid mixture includes an amount of suspended solids and wherein the treatment vessel includes a channel having a first end and a second end, the source of ultrasonic energy is positioned toward the first end, a length of the channel between the first and second ends corresponding to the amount of suspended solids in the fluid mixture during operation.

62. The apparatus of claim 51 wherein the treatment vessel includes a first channel, a second channel, and a manifold hydraulically connected to the first and second channels and to the inlet to direct a first portion of the fluid mixture to the first channel and a second portion of the fluid mixture to the second channel.

63. The apparatus of claim 51 wherein a molecular structure of a constituent of the fluid mixture has a resonant frequency and wherein the source of ultrasonic energy is configured to emit energy at and/or above the resonant frequency.

64. The apparatus of claim 51 further comprising an ozone source coupled to the treatment vessel to provide ozone to the fluid mixture during operation.

65. The apparatus of claim 51 wherein the ultrasonic energy source operatively coupled to the treatment vessel further comprises an ultrasonic energy source operatively coupled to the treatment vessel configured to transmit ultrasonic energy to the fluid mixture at a selected energy level and selected frequency.

66. An apparatus for removing a selected constituent from a fluid mixture, the apparatus comprising:
a treatment vessel having an inlet hydraulically connected to a source of the fluid mixture during operation, a channel configured to contain a flow of the fluid mixture, and an outlet downstream from the channel;
a pressure source in fluid communication with the treatment vessel to pressurize the fluid mixture as the fluid mixture passes through the treatment vessel;
an ultrasonic energy emitter coupled to the treatment vessel to transmit ultrasonic energy to the fluid mixture as the fluid mixture flows continuously through the treatment vessel, the source of ultrasonic energy being configured to continuously transmit ultrasonic energy to the fluid mixture at a rate and frequency sufficient to gasify and/or alter a chemical composition of the selected constituent;
a gas release chamber coupled to the outlet of the treatment vessel to receive a continuous flow of the fluid mixture; and
a vacuum source coupled to the gas release chamber to extract gas from the fluid mixture.
